(19) United States
(12) Patent Application Publication
(10) Pub. No.: US 2016/0064177 A1
(43) Pub. Date: Mar. 3, 2016

(54) X-RAY SOURCE AND IMAGING SYSTEM

(71) Applicant: SIEMENS AKTIENGESELLSCHAFT, München (DE)

(72) Inventors: Svetlana GOSSMANN, Erlangen (DE); Oliver HEID, Erlangen (DE); Timothy HUGHES, Wantage (GB)

(73) Assignee: SIEMENS AKTIENGESELLSCHAFT, München (DE)

(21) Appl. No.: 14/888,312

(22) PCT Filed: Mar. 7, 2014
(86) PCT No.: PCT/EP2014/054407

§ 371 Date: Oct. 30, 2015

(30) Foreign Application Priority Data
May 3, 2013 (DE) 10 2013 208 103.0

(51) Int. Cl. H01J 35/18; A61B 6/00

(52) U.S. Cl. CPC H01J 35/18; A61B 6/4035; H01J 2235/168

(57) ABSTRACT

An evacuable outer housing having at least one X-ray-permeable beam exit window, an electron source, an anode and a collector for catching electrons which penetrate the anode are included as an X-ray source. The collector is part of an electrical current circuit for applying a negative potential to the anode, and the radiation window is disposed such that X-ray radiation which exits from the anode at an angle of 130 degrees to 230 degrees to the electron beam direction can be coupled out through the radiation window. An imaging system includes such an X-ray, an arrangement to accommodate an object to be examined, and an X-ray detector.

### CROSS REFERENCE TO RELATED APPLICATIONS

This application is the U.S. national stage of International Application No. PCT/EP2014/054407, filed May 7, 2014 and claims the benefit thereof. The International Application claims the benefit of German Application No. 102013208103.0 filed on May 3, 2013, both applications are incorporated by reference herein in their entirety.

### BACKGROUND

Described below are an X-ray source having an evacuable outer housing with a beam exit window, an electron source for emitting electrons and an anode for generating X-radiation and an imaging system having such an X-ray source.

In known X-ray sources, electrons are accelerated inside an evacuable outer housing, a so-called X-ray tube, onto an anode, the material of which is suitable for converting energy of the accelerated electrons into X-radiation. The X-radiation is output from the X-ray source through an X-ray transparent exit window. When used in an imaging system, the radiation is then typically directed onto an object to be examined and subsequently measured by an imaging X-ray detector. Above all in medical imaging, the use of such systems is widespread. For the diagnostic examination of human body parts, it is generally desirable to achieve an image quality which is as high as possible with an X-ray dose which is as low as possible. To this end, maximally monochromatic X-radiation is advantageous, the radiation essentially consisting of characteristic X-radiation and only to a proportion which is as low as possible of bremsstrahlung distributed over a wide energy range.

U.S. Pat. No. 7436931 B2 describes an X-ray source for generating monochromatic X-radiation. In this case, a very thin anode is used, which is applied on an anode carrier made of a material with a low atomic number. The effect achieved by this is that characteristic X-radiation in a narrow energy range is essentially formed by the anode layer. Because of the small layer thickness of the anode and the low atomic number of the carrier, furthermore, little bremsstrahlung is emitted, so that only a small proportion of broadband X-radiation is generated by the source. One difficulty with the solution proposed in U.S. Pat. No. 7436931 B2, however, results from the high-energy electrons passing through the thin anode. These electrons are collected in the anode carrier, and the energy is dissipated by a coolant flowing through the carrier. One disadvantage in this case is the large generation of heat inside the anode carrier and the possibility of bremsstrahlung being produced in the anode carrier. Bremsstrahlung produces a continuous background in the resulting X-ray spectrum, which extends up to a cutoff energy that corresponds to the kinetic energy of the accelerated electrons. The proportion of characteristic monochromatic X-radiation in the overall spectrum and in the radiation dose is reduced by this effect. Because of the large generation of heat and the need for a coolant flow, this solution is furthermore inefficient in terms of thermal technology and mechanically elaborate.

### SUMMARY

An X-ray source generating maximally monochromatic X-radiation avoids the aforementioned disadvantages. Described below is an imaging system having such an X-ray source.

The X-ray source has an evacuable outer housing having at least one X-ray transparent beam exit window. It furthermore includes an electron source for emitting electrons along an electron beam direction, an anode for generating X-radiation, and a collector for collecting electrons passing through the anode. The collector is part of an electrical circuit for applying a negative potential to the collector in relation to a potential at the anode. The beam exit window is arranged in such a way that X-radiation which emerges from the anode at least in a subrange of an angle range of from 130 degrees to 230 degrees with respect to the electron beam direction can be output through the beam exit window.

The X-ray source described herein makes it possible to generate essentially monochromatic X-radiation, since in the anode mainly characteristic x-radiation is generated in a narrow energy range. The electrons passing through the anode furthermore contribute little to the formation of undesired bremsstrahlung, since these electrons are initially decelerated efficiently by the collector and then collected. The capture of the accelerated electrons by the collector is electrically efficient, and no additional coolant channel for dissipating the kinetic energy of these electrons passing through the anode film is required in the holder of the anode. Because of the electrical potential of the collector, which is negative relative to the anode during operation, the electrons lose some of their kinetic energy before they strike the material of the collector. In this way, the bremsstrahlung formed in the material of the collector is minimized. The collector prevents these electrons from reaching further components, in which they can generate bremsstrahlung, during operation of the X-ray source, and they are prevented from leaving the X-ray source. In particular, because of their efficient collection, these electrons do not interact with the outer housing of the X-ray source.

The beam exit window is arranged in such a way that X-radiation which emerges from the anode at least in a subrange of the angle range from 130 degrees to 230 degrees with respect to the electron beam direction can be output through this window. The output thus takes place on the side of the anode which faces toward the incident electron beam, in which case the X-radiation which is output through the window may cover an angle range of up to +/−50 degrees with respect to the backward direction of the electron beam. The effect achieved by this backward output is that the ratio of characteristic X-radiation to continuous bremsstrahlung is particularly high, since the bremsstrahlung has a substantially higher component in the direction of the electron beam, while the proportions of the characteristic X-radiation in the forward and backward directions are essentially symmetrical.

The imaging system has the X-ray source described herein, an arrangement for receiving an object to be examined, and an X-ray detector. The advantages of the imaging system are obtained in a similar way as the advantages specified for the X-ray source. In the field of medical imaging, the object to be examined may in this case be a human body, an animal body or a part of such a body. The arrangement for receiving the object to be examined is then, for example, a patient table or an arrangement for receiving a body part. The imaging system may, however, also be configured in order to measure components. In this case, the arrangement for receiving the object to be examined may be a holder for a component.

The advantages of the imaging system become particularly significant in medical imaging, since for the diagnostic examination of human body parts it is particularly important to achieve an image quality which is as high as possible, and therefore a medical diagnosis which is as accurate as possible, with a radiation exposure which is as low as possible. When using X-ray sources which are as monochromatic as possible, it is possible to achieve a particularly good image quality. The advantage of monochromatic X-ray sources is particularly great in the field of mammography and angiography, since in these processes body parts are examined for which it is necessary to image small differences in the attenuation of the X-radiation. When using monochromatic X-radiation, with comparable image quality, either the radiation exposure of the patient can be reduced, or the otherwise necessary use of X-ray contrast agent, which is harmful to health, can be avoided.

The X-ray source may additionally have the following features:

The collector may be configured to be thicker along the electron beam direction than the average penetration depth of the electrons for a kinetic energy of the electrons of 150 keV. For many X-ray sources, the maximum kinetic energy to which the electrons are accelerated in X-ray sources is in the range of up to 150 keV. If the collector is configured in such a way that in the range of this electron energy it is thicker than the average penetration depth of the electrons, then a substantial proportion of the electrons with this maximum energy will be captured by the collector during operation of the X-ray source. If the collector is furthermore, as is provided, brought to a negative potential during operation, then the electrons will be decelerated before entering the material of the collector, and correspondingly an even greater proportion of the electrons will be collected by the collector. The proportion of electrons collected by the collector is in this embodiment at least 1-1/e, and therefore more than 63%.

The material of the described collector may be an electrically conductive material, for example stainless steel and/or copper. The collector may have a thickness of at least 1 mm along the electron beam direction. The thickness is selected in such a way that the electrons reaching the collector with their remaining kinetic energy cannot substantially pass through the thickness of the collector.

The collector may have a depression in the electron beam direction. Such a depression is advantageous in order to reliably collect the accelerated electrons in the collector and prevent lateral escape of the electrons toward the outer housing of the X-ray source. The formation of a depression of the collector is expedient since a certain fraction of the electrons are scattered at the anode and therefore have their flight direction changed. A collector having a depression is particularly suitable for collecting as many scattered electrons as possible.

The described depression may be configured trapezoidally. As an alternative, the depression may also be configured rectangularly, in a U-shape or semicircularly. It may have a depth of at least 3 cm, and the depth may particularly advantageously be between 5 cm and 15 cm.

The beam exit window may be arranged in such a way that X-radiation which emerges from the anode at least in a subrange of an angle range of from 170 degrees to 190 degrees with respect to the electron beam direction can be output through the beam exit window. In this embodiment, only X-radiation which leaves the anode within an angle of +/−10 degrees to the backward direction of the electron beam is thus output. Because of this narrow angle range, an even better ratio of characteristic X-radiation to the perturbing continuous bremsstrahlung is achieved.

In another variant of this embodiment, the electron source may have a hole in a central region for X-radiation to be output to pass through. In particular, the electron source may be configured as an annular source. In the central region, the X-radiation to be output on the rear side can pass through the electron source and travel through this region from the anode to the beam output window. The beam exit window may then particularly advantageously be arranged in such a way that only X-radiation which emerges from the anode within an angle of from 175 degrees to 185 degrees with respect to the electron beam direction can be output through the beam exit window.

The X-ray source may include at least one control electrode for accelerating and/or focusing the electrons onto the anode. The X-ray source may also have a plurality of such control electrodes. The at least one control electrode may be an electrode having a circular cross section, and it may for example have the shape of one or more segments of a spherical surface. So that acceleration of the electrons takes place, the voltage of the control electrode should be higher than the voltage of the electron source.

The anode may have a metal layer, including a material that has an atomic number of at least 40 and the layer thickness of which is less than the average penetration depth of the electrons in the material of the metal layer for a kinetic energy of the electrons of 150 keV. The advantage of this embodiment is that a particularly high proportion of characteristic X-radiation is formed in a material with a relatively high atomic number. Particularly suitable materials are molybdenum with an atomic number of 42 and tungsten with an atomic number of 74. The advantage of the small layer thickness is that only a minimal amount of bremsstrahlung is generated in the metal film of the anode. The selection of the layer thickness depends on the anode material, because the penetration depth depends on the anode material properties. Advantageous layer thicknesses lie for example in the range of up to 10 μm, particularly advantageously in the range of up to 5 μm. A greater layer thickness is not required since the electrons passing through the anode are decelerated and captured by the collector.

The anode may have an anode carrier which includes a material that has an atomic number of at most 15 and the layer thickness of which is less than the average penetration depth of the electrons in the material of the anode carrier for a kinetic energy of the electrons of 150 keV. The selection of a light material for the anode carrier is advantageous because in this way little bremsstrahlung is generated in the anode carrier, since materials with a low atomic number have only a small interaction with the electrons. The anode carrier itself is used to hold the metal layer of the anode and to ensure mechanical stability. In the case of the carrying body as well, a thickness which is as small as possible contributes to avoiding undesired bremsstrahlung. The thickness of the holder may however be selected to be greater than the thickness of the metal layer, since in a material with a lower atomic number the interaction of electrons is less and therefore the average penetration depth for a particular kinetic energy is greater than for the metal layer. Here again, the relevant electron energy is dictated by the maximum acceleration voltage. Typically, an acceleration voltage of at most 150 kV is applied, which leads to a maximum kinetic energy of 150 keV.

The electrical circuit may be configured in such a way that the collector can be brought during an operation of the X-ray source to an electrical potential which is lower by at least one half than an electrical potential of the anode, the potentials of the collector and of the anode being defined in relation to the potential of the electron source, and in relation to this reference potential both are positive. The effect achieved by this potential difference is that the electrons which pass through the anode already lose a significant part of the energy in the field between the anode and the collector on their way from the anode to the collector.

The electron source may be a field-emission cathode or an incandescent cathode. A field-emission cathode is a so called cold cathode, in which electrons are typically emitted by a very high local field into the evacuated space of the X-ray source. In contrast thereto, in the case of an incandescent cathode the electrons are emitted from the cathode material into the evacuated space under the effect of high temperature.

The anode may be configured as a fixed anode, a rotating anode and/or a liquid anode. In the case of a fixed anode, the metal anode layer is held in a stationary holder. Conversely, a rotating anode includes a rotatably mounted, usually disk-shaped plate, which is rotated within the plane of the plate in such a way that the electron beam strikes different successive positions in the edge region of the plate, so that better heat dissipation and a longer lifetime of the metal anode layer are achieved. In the case of a liquid anode, an electrically conductive liquid is used as the anode layer, for example low-melting metals and alloys which contain gallium, indium and/or tin. The anode may also have a plurality of metal layers, which may for example, contain different materials. The metal layers may be arranged next to one another on a common carrying body. An X-ray source having a plurality of anode materials may be configured in such a way that, depending on the application, monochromatic X-radiation with different energy can be made available, depending on which of the anode materials is brought into the region of the electron beam.

The imaging system may additionally have a beam filter arranged between the beam exit window and the arrangement for receiving the object to be examined. Such a beam filter may contain a metal layer, for example of aluminum, rhodium, molybdenum, copper and/or tin, which is used to absorb the low-energy part of the continuous bremsstrahlung. This has the advantage that the object to be examined, in particular a body part of a patient, is not exposed to this filtered-out part of the X-ray spectrum. The low-energy part of the bremsstrahlung contains at most a very low proportion of the image information to be measured, since typically this part of the radiation is almost fully absorbed by the object to be examined, and no substantial part reaches the X-ray detector.

### BRIEF DESCRIPTION OF THE DRAWINGS

These and other aspects and advantages will become more apparent and more readily appreciated from the following description of an exemplary embodiment with reference to the accompanying drawings of which:

FIG. 1 is a schematic cross section of an X-ray source according to the exemplary embodiment;

FIG. 2 is a polar graph of the simulated angle dependency of the X-ray flux density of this X-ray source; and

FIG. 3 is a schematic side view of an imaging system having this X-ray source.

### DETAILED DESCRIPTION OF THE PREFERRED EMBODIMENT

Reference will now be made in detail to the preferred embodiments, examples of which are illustrated in the accompanying drawings, wherein like reference numerals refer to like elements throughout.

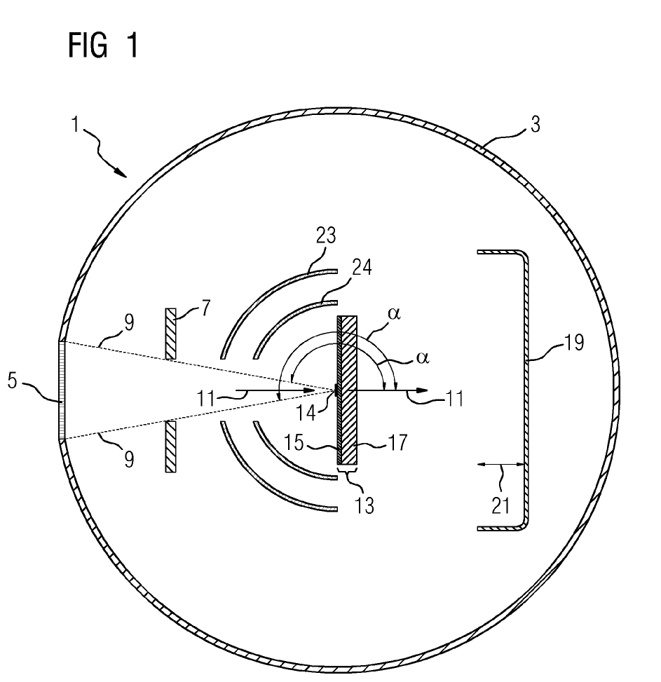

An X-ray source 1 according to an exemplary embodiment is shown as a schematic cross section in FIG. 1. This view shows a part of the outer housing 3, which can be closed in a gas-tight manner so that the interior of the X-ray source is evacuable. The formation of a vacuum is a prerequisite for the emission of electrons into this space and their acceleration in the direction of a predetermined position. The outer housing 3 is provided with a beam exit window 5, which is used to output the generated X-radiation 9 from the X-ray source 1. The beam exit window 5 is also sealed vacuum-tightly against the outer housing 3. A suitable material for the beam exit window 5 is, for example, beryllium.

Arranged inside the evacuable space are an electron source 7, an anode 13 and a collector 19, as well as in this example two control electrodes 23, 24. The electron source is in this case a cold field-emission cathode. It is configured annularly and is arranged in such a way that X-radiation 9 formed at the anode can reach the beam exit window 5 through the interior of this ring.

The electron source 7, the anode 13, the collector 19 and the control electrodes 23, 24 are part of an electrical circuit (not shown here). The electrons emitted into the vacuum by the electron source 7 are accelerated in the direction of the anode 13 by an electrical potential difference applied between the electron source 7 and the anode 13. In this example, the electron source 7 is at ground potential, and the anode 13 is at a voltage of 150 kV during operation. The two control electrodes 23, 24 are configured as parts of spherical surfaces, and they are used to accelerate and focus the electron beam emitted by the electron source 7 in the direction of the anode 13. In this example, the first control electrode 23 is at a potential of 10 kV and the second control electrode 24 is at a potential of 150 kV. The emitted electrons are thereby concentrated onto a focal spot 14 on the surface of the anode 13 and, in this example, strike the surface of the anode 13 perpendicularly along the electron beam direction 11.

In the exemplary embodiment shown, the anode 13 is a disk-shaped anode 13, which on the side facing toward the electron source has a metal layer 15 of 2 μm thick molybdenum, which is applied on an anode carrier 17. The anode carrier 17 may be a 15 μm thick diamond disk. In the thin molybdenum layer, a part of the energy of the accelerated electrons is converted into characteristic X-radiation of molybdenum. The emission of the characteristic X-radiation from the focal spot 14 of the electrons initially takes place isotropically in all spatial directions. The energy of the characteristic X-radiation lies at the energy of the K_{α} transitions of molybdenum at 17.4 keV and the K_{β} transitions at 19.6 keV. By the emission of characteristic X-radiation, quasi-monochromatic radiation in this energy range is thus made available. Another suitable anode material is for example tungsten, which is suitable for generating quasi-monochromatic X-radiation in the range of from 59 keV to 67 keV.

The small layer thickness of the metal layer 15 is selected in such a way that it is less than the average penetration depth in this material of electrons accelerated to an energy of 150 keV. A minimum layer thickness of several μm is necessary so that a sufficient proportion of electrons can interacts with the molybdenum in order to generate characteristic X-radiation. At the same time, it is desirable to keep the layer thickness as small as possible so that the generation of continuous bremsstrahlung is minimized. Because of the small layer thickness, a large proportion of the accelerated electrons is not absorbed by the molybdenum layer 15, but enters the anode carrier 17. In this example, the anode carrier 17 may be a diamond disk, so that only a small interaction with the accelerated electrons takes place because of the low atomic number of the carrier material. The thickness of the anode carrier 17 is also configured to be so small that a large proportion of the accelerated electrons passes through the anode carrier 17. This remaining fraction continues to move along the electron beam direction 11 on to the collector 19. The function of the collector 19 is to decelerate and collect the remaining electrons. So that the electrons can be decelerated, during operation of the X-ray source 1 the collector 19 is at an electrical potential which is negative in relation to the potential of the anode 13. In this exemplary embodiment, the collector 19 is at a potential of 30 kV, so that the electrons are decelerated to a small fraction of their original kinetic energy on the path between the anode 13 and the collector 19.

The material of the collector 19 is configured in such a way that a predominant fraction of the electrons is collected in the collector 19. In this example, the collector 19 is made of stainless steel. The thickness of the collector 19 in the electron beam direction 11 is also configured in such a way that a maximally high absorption of the electrons takes place, the wall thickness being 4 mm in this example.

The geometrical arrangement of the anode 13, the electron source 7 and the beam exit window 5 is configured in this example in such a way that X-radiation formed at the anode 13 can be output through the beam exit window 5 in an advantageous range of angles α₁ with the electron beam direction 11 of between 170 degrees and 190 degrees. In this angle range α₁, the X-radiation can pass through the opening of the electron source 7. As an alternative, the geometry of the X-ray source 1 may also be configured in such a way that radiation in a larger angle range a between 130 degrees and 230 degrees is output through the beam exit window 5. In this case, the electrons may also be guided from an electron source arranged laterally with respect to the beam path by control electrodes in the direction of the anode, so that the electron source does not lie in the region of the radiation to be output. Alternatively, the opening in the central region of the electron source 7 may be selected to be so large, or the electron source 7 may be arranged so near to the anode 13, that radiation in the angle range a between 130 degrees and 230 degrees is also output through the beam exit window 5.

The effect achieved by the specified geometry of the output and the selected range of angles α₁ of the output X-radiation with the electron beam direction 11 is that the radiation emerging from the X-ray source has a greatest possible proportion of characteristic X-radiation 25 and a proportion of bremsstrahlung 27 which is as low as possible, i.e. the X-radiation is essentially quasi-monochromatic.

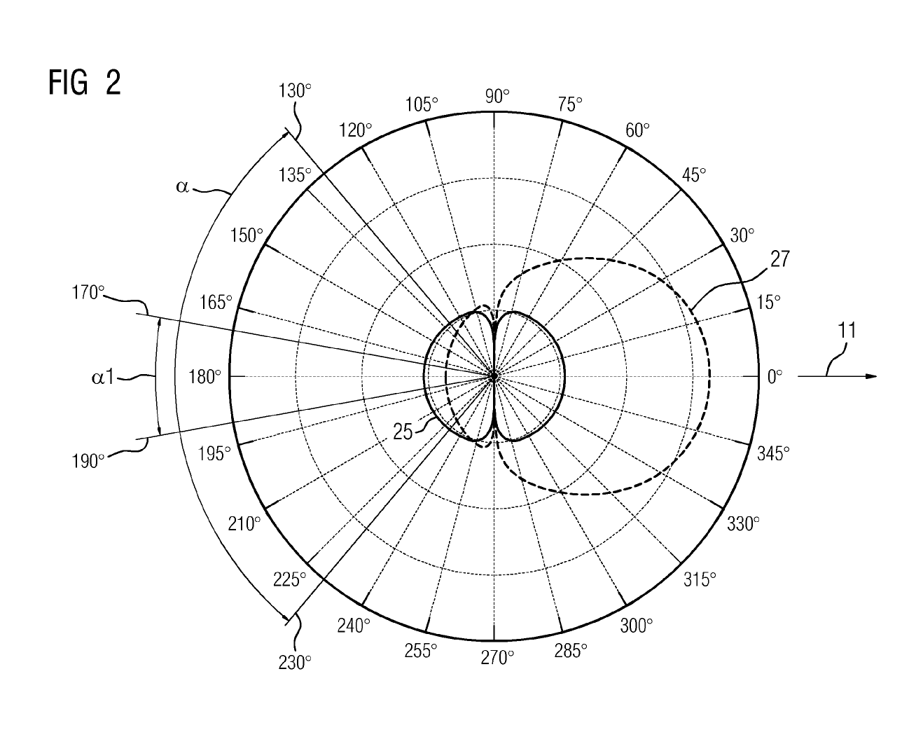

The effect of the output geometry on the composition of the X-radiation is illustrated in FIG. 2. FIG. 2 shows comparatively the stimulated X-ray flux density for the characteristic X-radiation 25 and for the bremsstrahlung as a function of the angle with the electron beam direction 11 for the above-specified materials and layer thicknesses of the anode 13 of the exemplary embodiment. For the simulation of the radiation intensities, for all angles the passage of the radiation through a beam filter 35 formed of a 30 μm thick molybdenum layer was additionally assumed. The simulation results in FIG. 2 show clearly that in an angle range between 90 degrees and 270 degrees, i.e. in the forward direction of the electron beam, for all angles the bremsstrahlung 27 is essentially more intense than the characteristic X-radiation 25.

In the backward direction, conversely, in a particular angle range the flux density of the characteristic X-radiation 25 dominates the bremsstrahlung 27. In an angle range a between 130 degrees and 230 degrees, the flux density of the characteristic X-radiation 25 is significantly higher, so that the continuous bremsstrahlung 27 forms only a weak background below the characteristic emission bands. The angle range α₁ between 170 degrees and 190 degrees is particularly advantageous for the generation of quasi-monochromatic radiation. The favorable intensity relationships, illustrated by the simulation, between characteristic X-radiation 25 and bremsstrahlung 27 are influenced not only by the selection of the output angle α, but also crucially by the materials and thicknesses of the anode, as well as by the possibility of collecting the electrons passing through the anode in the collector 19 and thereby minimizing the additional emission of bremsstrahlung.

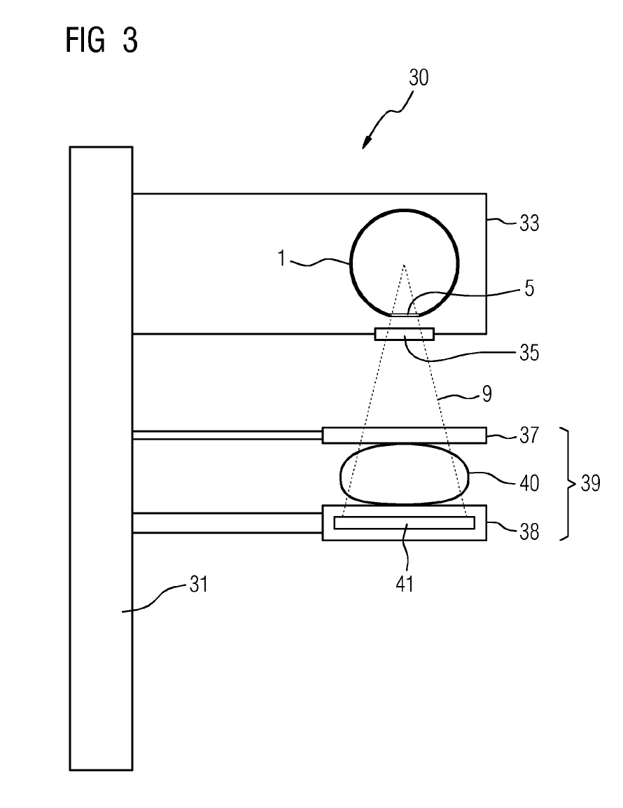

FIG. 3 shows a schematic cross section of an imaging system 30 having an X-ray source 1 according to the above-described exemplary embodiment. The imaging system 30 is in this case a mammography device, which is used for radiological examination of the female breast. For mammography, the use of maximally monochromatic X-radiation is particularly desirable, since in this examination method the imaging of very weak soft tissue contrasts in very small spaces is of primary importance. Here, an extremely high image quality is required above all for the detection and diagnosis of breast tumors. On the other hand, the female breast is very susceptible to the negative effects of ionizing radiation. Since mammography is also used as a screening method, in this case it is particularly important to optimize the achieved image quality in relation to the X-ray dose used.

The imaging system 30 contains the X-ray source 1 shown in detail in FIG. 1, Which is suspended from a carrying column 31 by a carrying arm 33. A height-adjustable carrier 38 is mounted on the carrying column 31, and a likewise height-adjustable compression plate 37, which together form an arrangement 39 for receiving an object 40 to be examined, here the female breast. The quasi-monochromatic X-radiation 9 generated by the X-ray source 1 is output through the beam exit window 5 and passes through a beam filter 35 arranged below the X-ray source 1. The beam filter 35 may be a 30 μm thick molybdenum layer, and it is used to filter out a part of the low-energy continuous bremsstrahlung before the X-radiation 9 strikes the breast 40 to be examined. Sequentially, the X-radiation 9 passes through the compression plate 37 onto the compressed breast 40. The fraction of the X-radiation 9 passing through the breast 40 is measured by an X-ray detector 41, in this case arranged inside the carrier 38 and processed by downstream readout electronics (not shown here) to form a diagnostically usable X-ray image.

A description has been provided with particular reference to preferred embodiments thereof and examples, but it will be understood that variations and modifications can be effected within the spirit and scope of the claims which may include the phrase “at least one of A, B and C” as an alternative expression that means one or more of A, B and C may be used, contrary to the holding in Superguide v. DIRECTV, 358 F3d 870, 69 USPQ2d 1865 (Fed. Cir. 2004).

1-15. (canceled)

16. An X-ray source, comprising:
an anode generating X-ray radiation;
an electron source emitting electrons along an electron beam direction;
an evacuable outer housing having at least one beam exit window, transparent to X-rays, arranged such that X-radiation emerging from the anode at least in a subrange of an angle range from 130 degrees to 230 degrees with respect to the electron beam direction is output through the beam exit window; and
an electrical circuit, including a collector collecting electrons passing through the anode, applying a negative potential to the collector in relation to a potential at the anode.

17. The X-ray source as claimed in claim 16, wherein the collector is thicker along the electron beam direction than an average penetration depth of the electrons in a material of the collector for the electrons having kinetic energy of 150 keV.

18. The X-ray source as claimed in claim 17, wherein the material of the collector is at least one of stainless steel and copper and has a thickness of at least one millimeter along the electron beam direction.

19. The X-ray source as claimed in claim 16, wherein the collector has a depression in the electron beam direction.

20. The X-ray source as claimed in claim 19, wherein the depression has at least one of a trapezoidal shape and a depth of at least 3 centimeters. 21 (New) The X-ray source as claimed in claim 16, wherein the subrange of the X-radiation emerging from the anode and passing through the beam exit window is from 170 degrees to 190 degrees with respect to the electron beam direction.

22. The X-ray source as claimed in claim 21, wherein the electron source has a hole in a central region through which the X-radiation passes towards the beam exit window.

23. The X-ray source as claimed in claim 16, having at least one control electrode controlling at least one of acceleration and focus of the electrons onto the anode.

24. The X-ray source as claimed in claim 16, wherein the anode has a metal layer formed of a material that has an atomic number of at least 40 and a layer thickness of less than an average penetration depth of the electrons in the material of the metal layer for the electrons having kinetic energy of 150 keV.

25. The X-ray source as claimed in claim 16, wherein the anode has an anode carrier formed of a material that has an atomic number of at most 15 and a layer thickness of less than an average penetration depth of the electrons in the material of the anode carrier for the electrons having kinetic energy of 150 keV.

26. The X-ray source as claimed in claim 16, wherein during operation of the X-ray source the collector has an electrical potential lower by at least one half of the electrical potential of the anode.

27. The X-ray source as claimed in claim 16, wherein the electron source is one of a field-emission cathode and an incandescent cathode.

28. The X-ray source as claimed in claim 16, wherein in the anode is one of a fixed anode, a rotating anode and a liquid anode.

29. An imaging system for an object to be examined, comprising:
an anode generating X-ray radiation;
an electron source emitting electrons along an electron beam direction;
an evacuable outer housing having at least one beam exit window, transparent to X-rays, arranged such that X-radiation emerging from the anode at least in a subrange of an angle range from 130 degrees to 230 degrees with respect to the electron beam direction is output through the beam exit window;
an electrical circuit, including a collector collecting electrons passing through the anode, applying a negative potential to the collector in relation to a potential at the anode;
an arrangement receiving the object to be examined; and
an X-ray detector.

30. The imaging system as claimed in claim 29, further comprising a beam filter arranged between the beam exit window and said arrangement receiving the object to be examined.
